(19) United States
(12) Patent Application Publication
(10) Pub. No.: US 2011/0262456 A1
(43) Pub. Date: Oct. 27, 2011

(54) METHOD OF TREATING ISCHEMIA REPERFUSION INJURY

(76) Inventor: Rekha Bansal, Twinsburg, OH (US)

(21) Appl. No.: 13/129,230

(22) PCT Filed: Nov. 16, 2009
(86) PCT No.: PCT/US09/64572

§ 371 Date: Jun. 21, 2011

Related U.S. Application Data
(60) Provisional application No. 61/114,787, filed on Nov. 14, 2008.

(51) Int. Cl. A61K 39/395; A61P 9/10

(52) U.S. Cl. 424/158.1

(57) ABSTRACT

A method of treating an ischemic reperfusion injury of a subject comprising administering to the subject a therapeutically effective amount of at least one of an adenosine receptor agonist or a protein kinase C activator and a therapeutically effective amount of a complement inhibitor.

### RELATED APPLICATION

This application claims priority from U.S. Provisional Application No. 61/114,787, filed Nov. 14, 2008, the subject matter, which is incorporated herein by reference.

### BACKGROUND OF INVENTION

Approximately 6 million people worldwide experience atrial fibrillation (AF), a condition in which the heart beats at two to three times its normal rate. Although AF is the most common of these arrhythmias, paroxysmal supraventricular tachycardia (PSVT) is also a relatively frequent occurrence with approximately 89,000 new cases being diagnosed on an annual basis. Overall, it is estimated that PSVT is present in 570,000 individuals and accounts for nearly 30,000 hospitalizations in the US.

It has been shown that ischemic preconditioning has a protective effect on the heart. Recently selected A2b agonist have been discovered and shown to provide similar results as that obtained due to pre-conditioning. These studies tested AMP579, CGX-1051 and bradykinin which activate p42/44 MAP kinases (ERKs), Akt or both. 5′-N-ethylcarboxamidoadenosine NECA has been used to study the A2b adenosine receptor (AR), although it is non-selective. A series of NECA derivatives has recently been reported to be modestly A2b AR selective as measured with a cyclic AMP assay at the A2b AR and binding assays at other AR subtypes. Several classes of non-nucleoside agonists have also been reported recently. The first highly potent agonist for the A2b AR is actually a non-nucleoside agonist. Based on the structure-activity relationship studies, the first selective A2b AR agonist, BAY-60-6583, has been developed. This class of compounds also display a remarkable agonistic-antagonistic profile at the A1 AR. All adenosine agonists A1, A2a, A2b and A3 have been shown to be involved with cardiovascular ischemia in multiple studies.

### SUMMARY OF THE INVENTION

The present invention relates to a method of treating an ischemic reperfusion injury of a subject. The method includes administering to the subject a therapeutically effective amount of at least one of an adenosine receptor agonist and/or protein kinase C activator and a therapeutically effective amount of a complement inhibitor. The therapeutically effective amount of the adenosine receptor agonist and/or protein kinase C activator can be an amount effective to mitigate ischemic damage of the reperfused tissue. The therapeutically effective amount of complement inhibitor can be an amount effective to inhibit complement activation. In one example, the adenosine receptor agonist can include an A2b adenosine receptor agonist. In another example, complement inhibitor can be a complement inhibiting antibody or fragment thereof.

### BRIEF DESCRIPTION OF THE DRAWINGS

FIG. 1 illustrates a plot of the infarction size as a percent of risk zone for control rabbit hearts and rabbit hearts treated with NECA.

FIG. 2 illustrates a plot of the infarction size versus risk zone for control and NECA treated rabbit hearts.

FIG. 3 illustrates MoAb⁷¹⁻¹¹⁰ inhibits C3a formation during extracorporeal circulation of whole human blood. As described in example 13, MoAb⁷¹⁻¹¹⁰ at various doses was evaluated for inhibition of C3a formation in a tubing loop model of cardiopulmonary bypass. FIG. 3 demonstrates dose dependent inhibition of C3a formation with complete inhibition observed at ˜10 μg/ml.

FIG. 4 illustrates MoAb⁷¹⁻¹¹⁰ inhibits C5a Formation during Extracorporeal Circulation of Whole Human Blood. Plasma from the tubing loop method was also evaluated for C5a formation as describe in example 2. FIG. 4 demonstrates that MoAb⁷¹⁻¹¹⁰ inhibits C5a formation dose dependently with complete inhibition observed at ˜10 μg/ml.

FIG. 5 illustrates MoAb⁷¹⁻¹¹⁰ inhibits C5b-9 formation during extracorporeal circulation of whole blood as evidenced by the Hemolysis Assay. C5b-9 is the terminal component of the complement cascade and is known as the membrane attack complex. According to the FIG. 5, treatment of MoAb⁷¹⁻¹¹⁰ dose dependently inhibits C5b-9 formation with complete inhibition observed ˜10 μg/ml.

FIG. 6 illustrates MoAb⁷¹⁻¹¹⁰ inhibits neutrophil activation during extracorporeal circulation of whole human blood. Aliquots of blood from the tubing loop method were evaluated using flow cytometry and the appropriate cellular activation markers. Neutrophils were evaluated using CD15-FITC and CD11b-PE antibodies. MoAb⁷¹⁻¹¹⁰ effects on cellular activation were evaluated. This figure demonstrates dose dependent inhibition of MoAb⁷¹⁻¹¹⁰ on neutrophil activation measure via CD11b expression. According to the figure, MoAb⁷¹⁻¹¹⁰ demonstrates complete inhibition at ˜10-20 μg/ml. These results coincide with the C3a and C5a results should be the case because C3a and C5a are potent activations of neutrophils.

FIG. 7 illustrates MoAb⁷¹⁻¹¹⁰ inhibits monocyte activation during extracorporeal circulation of whole human blood. Using the same principles as neutrophils, monocyte were stained with CD14-FITC and CD11b-PE. Monocyte activation was evaluated via CD11b expression. Treatment of MoAb⁷¹⁻¹¹⁰ demonstrates dose dependent inhibition with complete inhibition observed at ˜10 μg/ml.

FIG. 8 illustrates MoAb⁷¹⁻¹¹⁰ inhibits platelet activation during extracorporeal circulation of whole human blood. Using the same principles in FIGS. 3 and 4, platelet was measured using staining antibodies against platelet CD61-FITC and CD62P-PE. Platelet activation was evaluated by the level of CD62P expression. Treatment with MoAb⁷¹⁻¹¹⁰ produced results similar those obtained for neutrophils and monocytes. The figure demonstrates dose dependent inhibition of Platelet activation by MoAb⁷¹⁻¹¹⁰ with complete inhibition observed at ˜10 μg/ml.

FIG. 9 is a graph showing the effects of A2b agonist and complement inhibitor on infarct size.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this invention belongs.

As used herein, the term “complement inhibitor” refers to an agent, such as a small molecule, polypeptide, polynucleotide, or antibody that is capable of substantially reducing, inhibiting, blocking, and/or mitigating the activation complement in a subject.

As used herein, the term “subject” refers to any organism including, but not limited to, human beings, pigs, rats, mice, dogs, goats, sheep, horses, monkeys, apes, rabbits, cattle, etc.

As used herein, the terms “treatment,” “treating,” or “treat” refers to any specific method or procedure used for the cure of, inhibition of, reduction of, elimination of, or the amelioration of a disease or pathological condition (e.g., ischemic reperfusion injury) including, for example, preventing ischemic damage of a subject's heart upon reperfusion of the heart following a cardiac bypass.

As used herein, the term “effective amount” refers to a dosage of a complement inhibitor, adenosine receptor agonist, and/or PKC activator administered alone or in conjunction with any additional therapeutic agents that are effective and/or sufficient to provide treatment of ischemia damage and/or complement activation. The effective amount can vary depending on the subject, the disease being treated, and the treatment being effected.

As used herein, the term “therapeutically effective amount” refers to that amount of a PKC activator, AR agonists, and/or complement inhibitor administered alone and/or in combination with additional therapeutic agents that results in amelioration of symptoms associated with ischemic reperfusion injury or disorder associated with ischemic reperfusion injury and/or results in therapeutically relevant effect. By way of example, a “therapeutically effective amount” may be understood as an amount of PKC activator, AR agonists, and/or complement inhibitor required to reduce ischemic reperfusion injury in a subject.

As used herein, the terms “parenteral administration” and “administered parenterally” refers to modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the terms “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

As used herein, “Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

As used herein, the term “pharmaceutically acceptable salt” includes salts of compounds derived from the combination of the compound and an organic or inorganic acid or base.

The present invention relates generally to methods of treating an ischemic reperfusion injury in a subject by administering to the subject a therapeutically effective amount of at least one of an adenosine receptor (AR) agonist and/or protein kinase C (PKC) activator in combination with a therapeutically effective amount of a complement inhibitor. In one example, the adenosine receptor agonist can be an agent that prevents ischemic damage to the heart and the complement inhibitor can be an agent that inhibits or reduce surface induced activation of the alternative complement pathway. In another example, the method can include administering a mixture of an A2b AR agonist (i.e., A2b agonist) and an inhibitor of the alternative complement pathways to a subject to protect the heart from ischemia reperfusion injury during bypass.

It is known that (PKC) and A2b agonists can prevent the heart from ischemic damage. Complement inhibitors, such as those that prevent C3a and C5a formation via the alternative pathway (AP), are capable of inhibiting the AP and can be used in combination with AR agonist and/or the protein kinase C activators to prevent reperfusion injury along with ischemic damage. The adenosine receptor agonist and/or PKC can protect tissue from ischemic damage and the complement inhibitor can prevent complement and cellular activation induced via the artificial surfaces of the dead tissue and bypass circuit used during surgery. Advantageously, the administration to a subject of the adenosine agonist and/or PKC activator in combination with the complement inhibitor prior to and/or during bypass can substantially reduce ischemic reperfusion damage compared to the administration of either the adenosine agonist and/or PKC activator or complement inhibitor alone.

The method of the present invention can be used in clinical situations where PKC activators, AR agonists, complement inhibitors are known to play mutually exclusive roles in the disease. For example, in a clinical situation, an individual experiencing chest pain is examined and found to have a blood clot occluding the artery/vein. As a result of the blockade, the heart muscle does not receive blood and undergoes ischemia. When the clot is removed via mechanical/chemical means, the blood flow is restored but the ischemic/apoptotic tissue does not recover. Using experimental models of rabbit heart ischemia, it was found that PKC activators or adenosine agonists, such as A2b agonists, can protect heart damage. These experiments utilized BAY-60-683 (Bayer Health care) and 5′-N-ethylcarboxamidoadenosine (NECA) to demonstrate greater than 80-85% protection using rabbit models of cardiac ischemia.

We have shown, for example, in International Patent Application No. PCT/US2008/068530, which is incorporated herein by reference in its entirety, that blood circulating through the clinical bypass circuit or plastic tubing carries activated complement anaphylatoxins and activated neutrophils, monocytes, and platelets. Such extra-corporeal procedures are routinely employed for creating a bypass around the ischemic artery in cardiac ischemia, cardiac valve repairs and other cardiac surgical procedures. As a result of such extra-corporeal procedures, the patient is constantly receiving blood from the extracorporeal circuit, which contains activated inflammatory cells and activated platelets. Because the heart during the surgical procedures is kept with very little amount of blood, it nearly mimics a situation of no blood flow through the heart. Following the cardiac procedure, the blood circulates through the ischemic heart pouring activated neutrophils, monocytes, platelets and their respective inter-cellular aggregates causing more damage to the heart. This invention proposes that in the presence of a complement inhibitor, which is responsible for the inhibition of AP and cellular activation, the secondary damage to the heart will be near minimal.

In an aspect of the invention, the AR agonists can be A1, A2a, A2b, and A3 AR agonists. One example of an AR agonist is adenosine. Adenosine is the major endogenous agonist. Adenosine is similarly potent at A1, A2A and A3, but weak at the A2B.

Numerous other selective adenosine agonists are under development for potential therapeutic applications and some are already in the late phases of clinical trials, although none has yet received regulatory approval except for adenosine itself. Mainly, adenosine agonists are being developed for the following conditions: cardiac arrhythmias, neuro-pathic pain, myocardial perfusion imaging, inflammatory diseases and colon cancer.

Examples of selective A1 agonists include: CPA, CCPA, CHA and (S)-ENBA. CPA, CCPA and CHA are very selective at the murine A1. At human A1, these molecules only show a modest selectivity (10-30-fold over A3). (S)-ENBA is selective for the A1 in both humans and rats. CGS21680 and DPMA are selective at the murine, but not the human, A2A, and they are also fairly potent at the human A3. IB-MECA and C1-IB-MECA are potent at both human and rat A3, but a certain degree of species-dependent selectivity may also be found. A selective A1 agonist, GR-79236, has been in clinical trial for the treatment of pain, however, it was discontinued due to cardiovascular side effects. Phase II clinical trials of another A1 agonist, GW-493838 for the treatment of neuropathic pain has been completed.

An example of a selective A2a agonist is BVT.115959, which is now under Phase II clinical trials for diabetic neuropathic pain. A2a is abundant in coronary blood vessels. Based on preclinical animal work, three selective A 2A agonists, regadenoson, binodenoson and apadenoson, have been in Phase III studies as pharmacologic stress agents. Despite the high A2a selectivity of binodenoson and regadenoson in preclinical studies, subjective side effects attributable to other AR subtypes were still observed in human studies and although they are slightly lower than adenosine. Apadenoson was reported to be more selective than the other two agonists that entered Phase III trials earlier. Other A2a agonists have been developed and shown to inhibit multiple manifestations of inflammatory cell activation including production of superoxide, nitric oxide, TNF-α, IL-12, IL-10 and VEGF. A2a agonists are also vasodilators, but the inhibition of inflammation occurs at low doses that produce few or no cardiovascular side effects. Through A2a activation, adenosine can inhibit T-cell activation, proliferation, and production of inflammatory cytokines and enhance the production of anti-inflammatory cytokines.

An example of a selective adenosine A2b agonist is BAY-60-6583 (Bayer). BAY-60-6583 has been shown to be effective in reduction of the infarct size by application after the onset of cardiac ischemia in a rabbit infarct model.

5′-N-ethylcarboxamidoadenosine (NECA) has been used to study the A2B. A series of NECA derivatives has recently been reported to be modestly A2B selective as measured with a cyclic AMP assay at the A 2B and binding assays at other subtypes.

An example of an A3 agonist is IB-MECA. IB-MECA is an oral drug that has been successfully used in animal models and human Phase II rheumatoid arthritis trials to test the concept of targeting the A 3 for the treatment of inflammatory diseases. Phase I studies in healthy volunteers, as well as the Phase IIa clinical studies in RA human patients have demonstrated this drug has a favorable safety profile. Recent interim analysis of Phase IIa pilot study data indicates that another A3 agonist, CF101 (Can-Fite), has disease-modifying anti-inflammatory activity in RA patients failing methotrexate therapy. Can-Fite is now completing Phase IIb clinical trials of CF101 in RA. CF502, a more selective A3 agonist, is in preclinical development for RA. Certain A3 agonists Cladribine and cordycepin are used in the clinic for the treatment of Leukemia. IB-MECA (30 and 60 μM) and C1-IB-MECA (10-30 μM) induced apoptosis in HL-60 human promyelocytic leukemia cells. However, low concentrations of the A 3 agonist C1-IB-MECA (10 nM or 1 μM) actually protected against suggesting that apoptosis in HL-60 cells induced by adenosine analogs are not through the A3.

Examples of PKC activators that can be used to treat an ischemia reperfusion injury are phorbol 12-myristate 13-acetate, 1-hexylindolactam-V10, 6,11,12,14-tetrahydroxy-abieta-5,8,11,13-tetraene-7-one, 8-octyl-benzolactam-V9, and BMS-214662. Other PKC activators that are used in treatment of an ischemia reperfusion injury can be used in the combination therapy in accordance with the present invention.

The complement inhibitor can include any small, molecule, polypeptide, polynucleotide, antibody, and/or peptidomimetic that substantially reduces, mitigates, inhibits and/or blocks the classical and/or alternative complement pathway. In one aspect of the invention the complement inhibitor can advantageously inhibit alternative complement activation without inhibiting classical pathway activation.

In an aspect of the invention, the complement inhibitor can include an anti-complement antibody that can inhibit the alternative and/or classical complement pathway. Examples of antibodies that can inhibit the alternative pathway include at least one of an anti-C3 antibody, anti-C3b antibody, anti-Ba antibody, anti-Bb antibody, anti-P antibody, anti-D antibody, anti-C5 antibody, anti-C5a antibody, anti-C6 antibody, anti-C7 antibody, anti-C8 antibody, and anti-C9 antibody.

The antibody can be raised in a mammal and be, for example, monoclonal, polyclonal, recombinant, monospecific, bispecific, dimeric, humanized, chimeric, single chain, human, bispecific, truncated or mutated. In an aspect of the invention, the antibody can be an IgG, F(ab′)2, F(ab)2, Fab′, Fab, scFv, truncated IgG, or recombinant antibody.

In another aspect of the invention, the anti-complement antibody is incapable of activating the alternative complement pathway and prevents activation of neutrophils, monocytes, basophils, lymphocytes, and platelets via the alternative pathway.

In a further aspect of the invention, the anti-complement antibody can reduce the level of properdin in the blood. The reduced levels of properdin in blood can decrease levels of C3a, C5a, Bb, C5b-9 as a result of decreased alternative complement pathway activation during extracorporeal circulation. The reduced levels of properdin can also reduce cellular activation in blood from the subject following extracorporeal circulation. The anti-properdin antibody can further reduce the levels of one or more components of the alternative complement pathway and/or one or more factors produced by action of one or more components of the alternative complement pathway. Exemplary, non-limiting examples of components and factors that are reduced include MAC (C5b-9), C3c, and anaphylatoxins, such as C3a and C5a, and the like. The anti-properdin antibody reduces the levels of one or more components of the alternative complement pathway and/or one or more factors produced by action of one or more components of the alternative complement pathway by at least about 20%, at least about 30%, at least about 40%, at least about 50%, at least about 60%, at least about 70%, at least about 80%, at least about 90%, or more, compared to the level of the component or factor in the absence of the subject antibody.

In another aspect of the invention, the anti-properdin antibody can bind properdin with high affinity, inhibit oligomerization of properdin, inhibit factor D mediated cleavage of factor B in a C3bB complex, not inhibit the classical complement pathway, prevent alternative complement pathway activation, inhibit C3a, C5a, and C5b-9 formation, Inhibit neutrophil, monocyte and platelet activation. Inhibit leukocyte platelet conjugate formation.

An example of an anti-properdin antibody in accordance with the present invention was isolated and structurally characterized as described in the International Patent Application No. PCT/US2008/068530. The Examples of the present application and International Patent Application No. PCT/US2008/068530 disclose an anti-properdin antibody identified as MoAb⁷¹⁻¹¹⁰ that is produced by the hybridoma cell line deposited under ATCC Accession Number PTA-9019. MoAb⁷¹⁻¹¹⁰ was found to inhibit alternative complement pathway activation. MoAb⁷¹⁻¹¹⁰ inhibits factor D cleavage of C3bB complex. C3b produced by the cleavage of C3 binds factor B to produce the C3bB complex. Properdin binding to the complex C3bB promotes factor D induced cleavage of factor B. Evidence comes from studies using properdin-depleted serum, which has no complement activity suggesting that C3bB complex cannot be formed and cleaved with factor D in the absence of properdin. MoAb⁷¹⁻¹¹⁰ prevents factor D cleavage of PC3bB complex.

Surprisingly, MoAb⁷¹⁻¹¹⁰ has no effect on classical pathway activation. MoAb71-110 is an alternative pathway specific antibody and binds to a region on properdin that is only involved in AP activation. Use of MoAb⁷¹⁻¹¹⁰ will keep the classical pathway (CP) intact for host defense. Therefore, the present invention provides a process of inhibiting the alternative complement activation without inhibiting the classical pathway activation in vitro and in vivo in a human or animal subject.

Administration of these therapeutic agents (i.e., the PKC activator, AR agonist, and complement inhibitor) in combination typically is carried out over a defined period (usually minutes, hours, days or weeks depending upon the combination selected). “Combinatorial therapy” or “combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example by administering to the subject an individual dose having a fixed ratio of each therapeutic agent or in multiple, individual doses for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissue. The therapeutic agents can be administered by the same route or by different routes. The sequence in which the therapeutic agents are administered is not narrowly critical.

The exact formulation, route of administration, and dosage for the PKC activator, AR agonist, and complement inhibitor can be chosen by the individual physician in view of the subject's condition. Formulation of pharmaceutical compounds for use in the modes of administration noted above are described, for example, in Remington's Pharmaceutical Sciences (18th edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified-Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y. U.S.A., 1999.

In one aspect of the invention the complement inhibitor and the PKC activator and/or the AR agonist can provided in a pharmaceutical composition. The mixture of the two drugs can be made in vitro or in vivo, can be made by synthetically attaching the AR agonist to the complement inhibitor.

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Effect of Anti-P Monoclonal Antibody on Ischemic Damage in Rabbit Heart

New Zealand White rabbits of either sex weighing nearly 3 Kg were anesthetized with intravenous sodium pentobarbital (30 mg/kg). Throughout the experiment, additional anesthesia was administered as needed (5-15 mg pentobarbital/15 min) A heating pad maintained rectal temperature between 38.5 and 39.5-C. Animals were intubated through a tracheotomy and ventilated with 100% O₂ with the aid of a mechanical ventilator. To administer drugs, a PE 50 catheter filled with heparinized saline was placed in an ear vein. After a left thoractomy, a prominent branch of the left coronary artery was surrounded with a suture (2-0 silk) to form a snare. The rabbits were allowed to stabilize for 15 min after surgery before the protocols were begun.

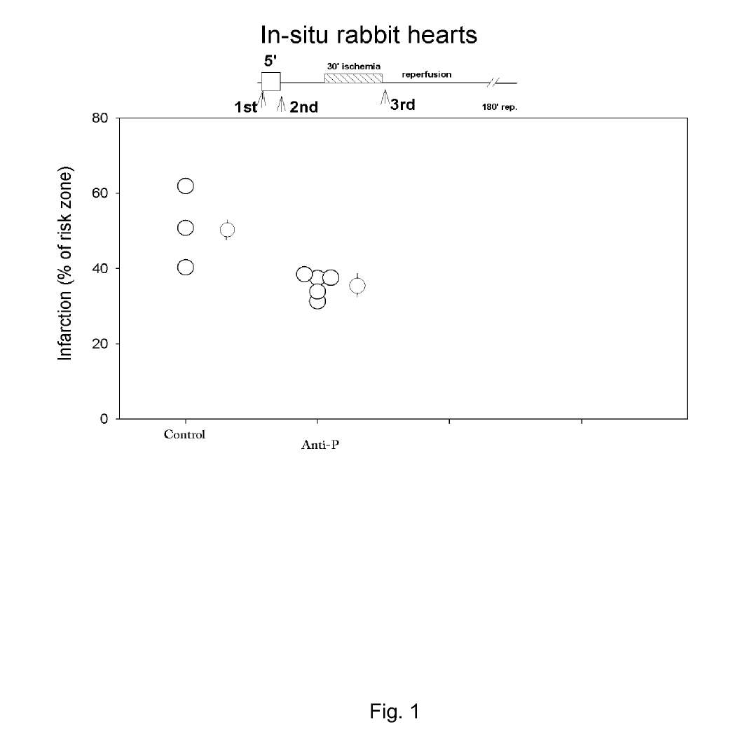

Hearts of 21 experimental groups experienced 30 min of regional ischemia (FIG. 1). In all hearts reperfusion following the occlusion lasted for 3 h. Control hearts had only the index occlusion followed by reperfusion. In all post-conditioned groups, four cycles of 30-s reperfusion/30-s occlusion were started immediately after release of the index ischemia. In drug-treated group NECA (2 Ag/kg), a non-selective but potent A2b adenosine agonist, was infused 5 min before the onset of reperfusion. After completion of the studies, all in situ hearts were excised and the aortic root was perfused with 0.9% saline. The coronary artery was re-occluded, and 2-9 Am diameter green fluorescent microspheres (Duke Scientific Corp., Palo Alto, Calif.) were infused into the perfusate to demarcate the ischemic zone as the area of tissue without fluorescence (region at risk). Hearts were weighed, frozen, and then cut into 2-mm-thick transverse slices. The slices were incubated in 1% triphenyltetrazolium chloride (TTC) in sodium phosphate buffer (pH 7.4) at 38-C for 8 min TTC stains the noninfarcted myocardium brick red indicating the presence of de-hydrogenase enzymes. The slices were then immersed in 10% formalin to enhance the contrast between stained (viable) and unstained (necrotic) tissue. The risk zone was revealed as the non-fluorescent region by illumination with UV light. The areas of infarct and risk zone were determined by planimetry of each slice and volumes were calculated by multiplying each area by its slice thickness and summing them for each heart. Infarct size is expressed as a percentage of the risk zone.

NECA was obtained from Sigma Chemical Co. (St. Louis, Mo.). The compound was dissolved in DMSO. The solution was then further diluted in 1.5 ml of 0.9% saline before administration to the rabbit. This amount of DMSO had no effect in control hearts. All data are expressed as mean TS.E.M. One-way analysis of variance (ANOVA) combined with Tukey's posthoc test was performed on baseline hemodynamic and infarct data. Temporal differences in hemodynamic variables in any given group were analyzed with one-way repeated measures ANOVA, and Tukey's post hoc test was used to examine differences between measurements at any given time point and baseline. Differences were considered to be significant if the p value was below 0.05.

New Zealand White rabbits of either sex weighing around 3 Kg were anesthetized with intravenous. We analyze is to plot infarct as a function of risk. Because the plot does not go through the origin that means that the risk zone is an independent determinant of the fraction of the risk zone that infarcts.

In that analysis, we do an ANOVA with covariates. The infarct becomes the dependent variable and the risk is the covariate. In FIG. 1, the open symbols depict the treated hearts. That analysis does show a difference at p=0.029. Thus, the infarct was statistically smaller in the treated group.

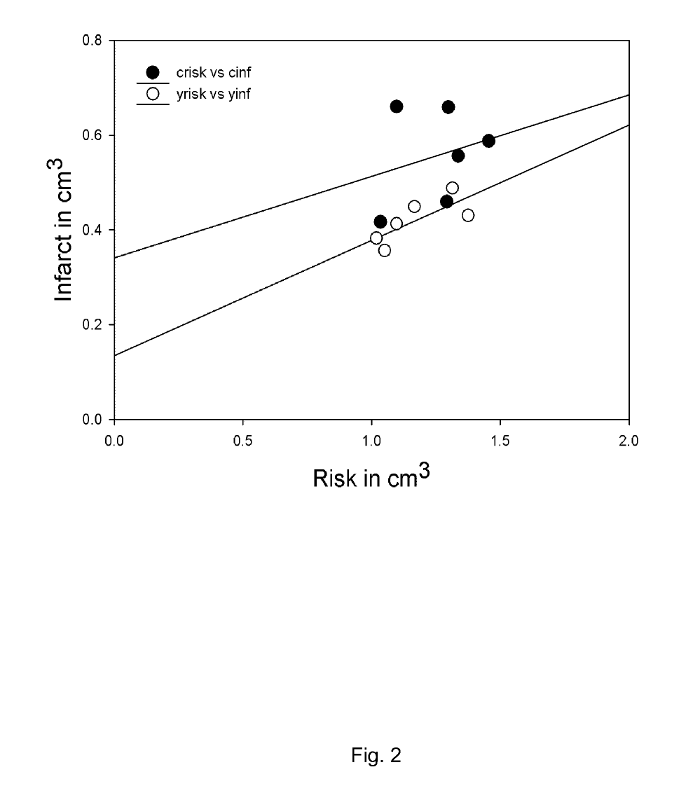

The total salvage is shown in FIG. 2. Also, the statistical tests warned us that the sample size was too small to reliably detect such a small difference with so much scatter.

### Example 2

### MoAb Inhibits Complement Activation in Whole Blood Model of Cardiopulmonary Bypass

The tubing loop model is a representative model to demonstrate alternative complement pathway mediated activation. The present model would evaluate AP activation in situations where blood comes in contact with the artificial surfaces. This model closely mimics the complement activation that is observed during extracorporeal circulation. The tubing loop experiment was performed by collecting fresh blood from a healthy donor using IRB approved guidelines. The blood was collected into 50 mL polypropylene conical tubes containing 5 units/ml of heparin (porcine mucosa) as an anticoagulant. The blood was added to the tubes preloaded with plasmalyte 148 containing various concentrations of MoAb⁷¹⁻¹¹⁰ . A 1.8 ml of this diluted blood containing the appropriate concentrations of the MoAb⁷¹⁻¹¹⁰ were added to poly vinyl chloride (PVC) tubings (PE 330; I.D., 2.92 mm; O.D., 3.73 mm; Clay Adams, N.J.). The tubings were closed into a loop and subjected to a vertical rotation for at least 2-3 hours at 37° C. After incubation, blood samples were transferred into 2.0 ml tubes. Blood aliquots for flow cytometry were acquired. The remaining blood samples were centrifuged (4000.times.g for 5 minutes at 4° C.) and the plasma were collected. The plasma samples were evaluated for complement activity utilizing C3a and C5b-9 kits (Quidel), C5a kits (BD-Pharmingen), and the rabbit erythrocyte lysis assay. The rabbit erythrocyte lysis was performed as described in previous examples with the only major difference being that tubing loop plasma samples were dilute 1:1 and then added to rabbit erythrocytes.

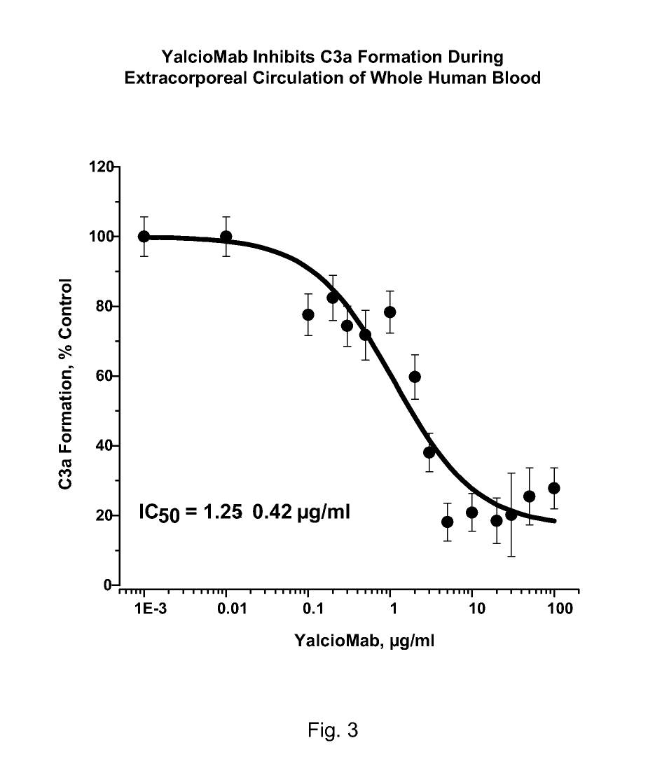

As a result of AP activation, C3-convertase forms, which cleaves C3 into C3a and C3b. C3a is a potent anaphylatoxin activates neutrophils, monocytes, and lymphocytes. During the tubing loop process, the alternative pathway is activated and results in the formation of C3 convertase, which causes the formation of C3a. FIG. 3 demonstrates that inhibition of the alternative pathway results in a dose dependent inhibition of C3a formation with complete inhibition observed at about 10 μg/ml.

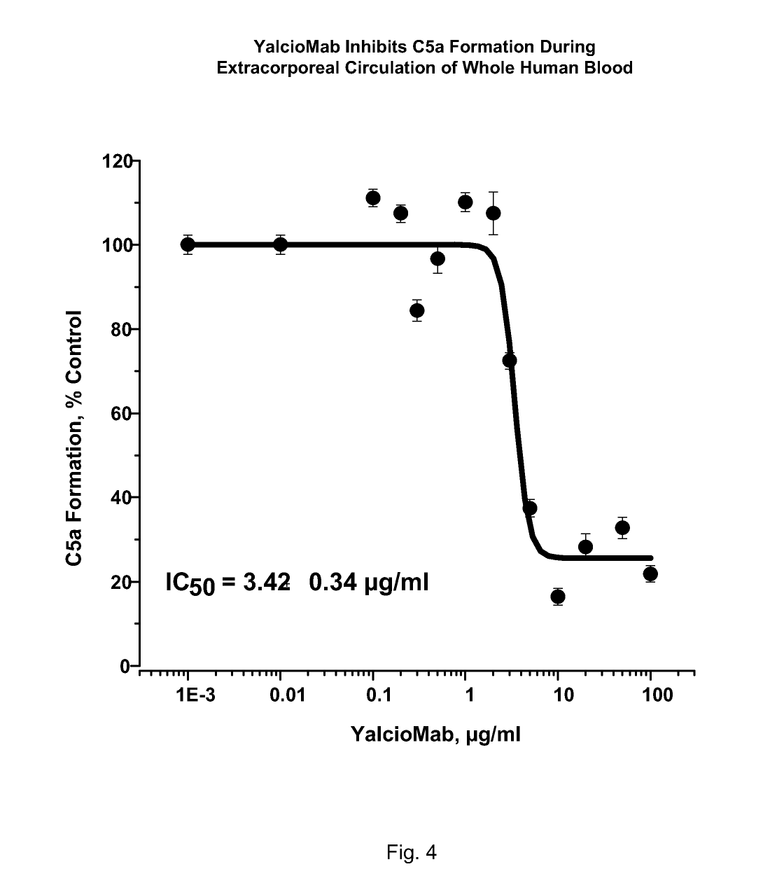

C5a is another potent anaphylatoxin that is responsible for activating many inflammatory cells. C5a is produced by C3-convertase cleavage of C5 into C5a and C5a. As demonstrated by FIG. 4, MoAb⁷¹⁻¹¹⁰ dose dependently inhibits C5a generation with complete inhibition at about 10 μg/ml. This number corresponds with the C3a results with complete inhibition at about 10 μg/ml which indicates that MoAb⁷¹⁻¹¹⁰ is able effectively inhibit C3 convertase function.

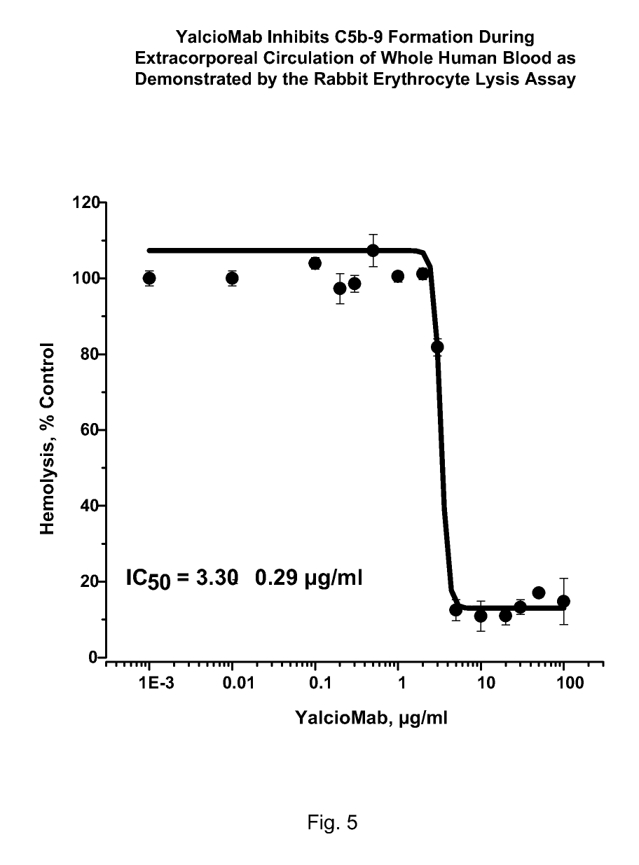

C5b-9 is terminal component of the complement cascade and functions as a lytic pore-forming complex that deposits onto membranes and causes their lysis. The hemolysis assay makes use of this concept and the formation of C5b-9 deposits onto these cells and causes their lysis. MoAb⁷¹⁻¹¹⁰ inhibits alternative pathway activity and thus prevents C5b-9 formation as demonstrated by FIG. 5. In this figure, MoAb⁷¹⁻¹¹⁰ is able to inhibit dose dependently C5b-9 formation with complete inhibit observed at ˜5-10 μg/ml. These numbers correspond with the values obtained from the C3a and C5a results.

### Example 3

### MoAb Inhibits Cellular Activation in Whole Blood Model of Cardiopulmonary Bypass

We have shown that MoAb⁷¹⁻¹¹⁰ inhibits inflammatory mediators C3a and C5a. These molecules are potent activators of neutrophils, monocytes, and platelets. Receptors for C3a and C5a are known to be present on each of these cell types. In a tubing loop model of cardiopulmonary bypass, we evaluated the ability of MoAb⁷¹⁻¹¹⁰ to inhibit cellular activation. Using the tubing loop method as described in the example above, we evaluated the effect of MoAb⁷¹⁻¹¹⁰ at various concentrations (0.5-100 μg/ml) on the effect of cellular activation. After the tubing loop process, an aliquot blood from the tubing loops was taken for flow cytometry analysis.

Aliquots of blood were stained with FITC-CD14 and PE-CD11b for monocytes, FITC-CD15 and PE-CD11b for neutrophils, and FITC-CD61 and PE-CD62P for platelets. Only 50 μl blood samples were labeled. Following a 20 min incubation at room temperature, 2 ml of FACS Lysing solution was added and the samples were incubated at room temperature for 20 min Samples were centrifuged for 5 min to pellet the cells. The supernatant is removed and the cells re-suspended in wash buffer (0.1% BSA in PBS/azide). The samples were re-centrifuged, the supernatant removed, and the cells re-suspended in 0.5 ml of 0.5% paraformaldehyde.

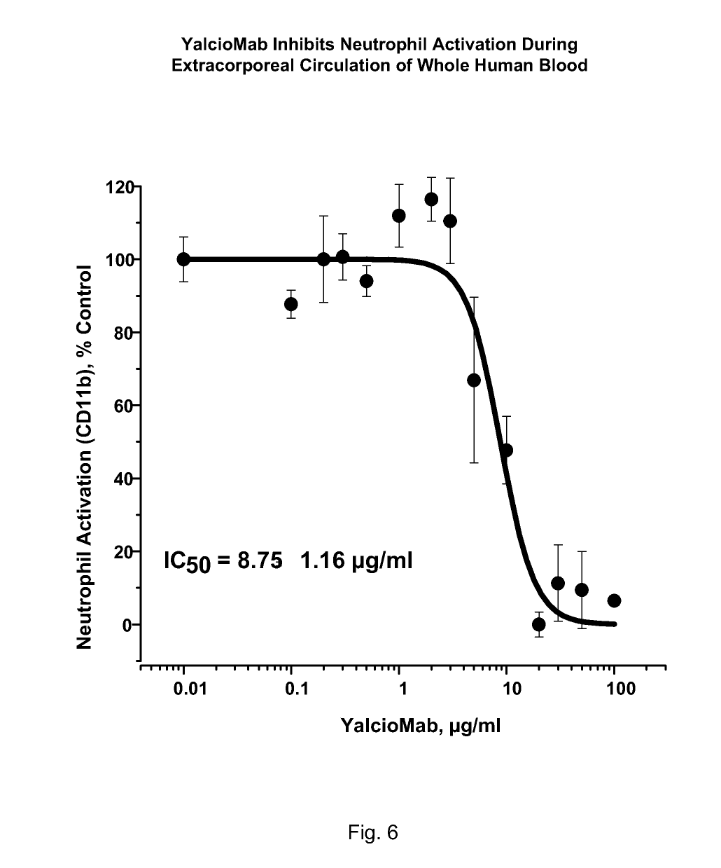

Neutrophils are potent inflammatory cells capable of releasing several deleterious components that mediate the inflammatory response. As described above, MoAb⁷¹⁻¹¹⁰ inhibits C3a and C5a formation during tubing loop. This anaphylatoxins are responsible for activation of neutrophils, monocytes, and platelets. Treatment with MoAb⁷¹⁻¹¹⁰ demonstrates dose dependent inhibition of neutrophil activation as measured via CD11b expression with complete inhibition observed at about 10-20 μg/ml (FIG. 6). This corresponds very closely with the results obtained for inhibition of C3a and C5a formation as.

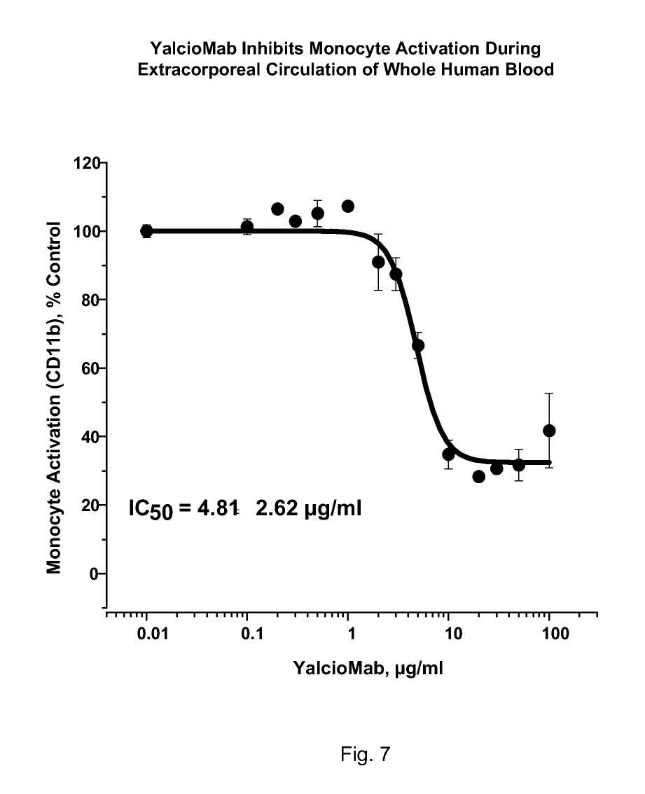

Monocytes are another important inflammatory cell that is responsible for the mediating the inflammatory response. They are most notably known for the production of TNF-alpha, one of the most potent inflammatory cytokines. Monocytes are activated via binding of C3a and C5a to their respective receptors. Monocytes were evaluated with flow cytometry as described earlier in this example. Treatment with MoAb⁷¹⁻¹¹⁰ demonstrates dose dependent inhibition of monocyte activation as measured via CD11b expression with complete inhibition observed at about 10 μg/ml (FIG. 7). This corresponds very closely with the results obtained for inhibition of C3a and C5a formation as well as the results from neutrophil inhibition.

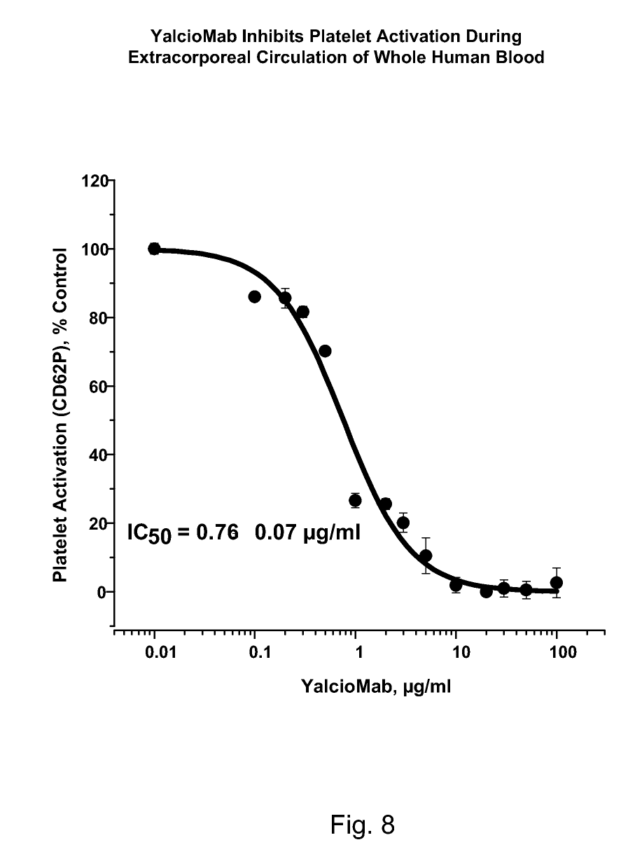

Platelets are activated via mechanisms similar to monocyte and neutrophil activation. However, platelets are not responsible for the inflammatory responses but are more important in regulating hemostasis. In cardiopulmonary bypass, platelet dysfunction leads to severe and complex bleeding complications. Prevention of platelet dysfunction is a critical aspect of developing therapeutics for cardiopulmonary bypass. Platelets were evaluated using flow cytometry described above using tubing loop samples. FIG. 8 demonstrates dose dependent inhibition of platelet activation measured via platelet activation marker CD62P. Complete inhibition is observed at about 10 μg/ml. These numbers are consistent with all other experiments.

### Example 4

### Effect of A2b Agonist and MoAb⁷¹⁻¹¹⁰ Combination on Ischemia Reperfusion Injury

A2b agonists have been shown to be effective in ischemic damage to the heart. MoAb71-110 inhibits alternative pathway complement activity. The total inhibition of infarct size by A2b appears to be in the range of 50% of the total infarct size. A2b agonist is not shown to inhibit the reperfusion damage. Reperfusion damage is caused by the flow of blood onto the ischemic tissue. MoAb⁷¹⁻¹¹⁰ inhibits the alternative complement pathway and hence is expected to inhibit reperfusion damage. In the rabbit model of ischemia reperfusion injury we propose to use the A2b agonist and MoAb⁷¹⁻¹¹⁰ to allow benefits from both ischemia and reperfusion. In a separate scenario, when a patient is attached to a CPB circuit, blood from the patient is circulated into the CPB circuit to allow oxygenation of the circulating blood and that the CPB circuit takes over the function of the lungs. We have previously shown that MoAb⁷¹⁻¹¹⁰ prevents AP activation in CPB circuit and that the antibody also prevents cellular activation. During the surgery, the heart contains very little amount of blood as surgeons operate on the ischemic heart. Following the surgery when blood returns to the heart from the CPB circuit, activated cells are poured into the ischemic heart and as a result, one can suffer from further ischemic insult.

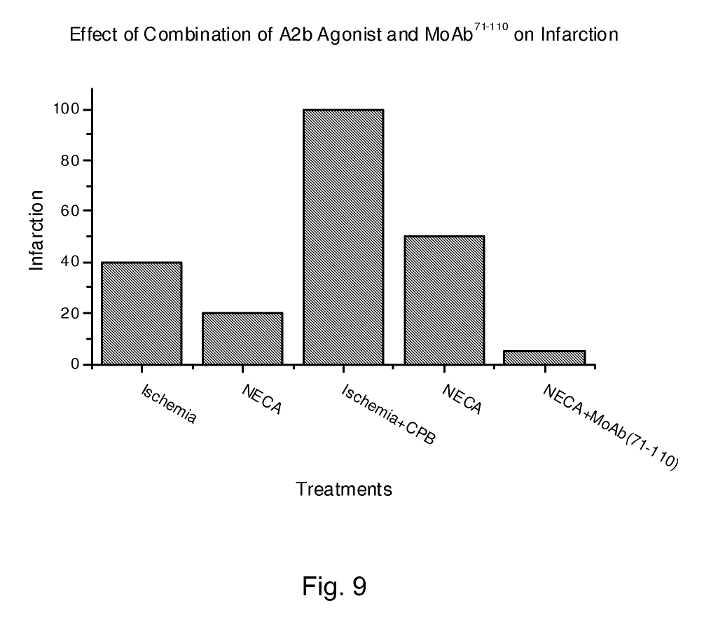

A therapy which consists of A2b agonist and MoAb⁷¹⁻¹¹⁰ would prevent both ischemia and reperfusion injury. Such combination therapy is expected to provide benefit in ischemia reperfusion. As shown in FIG. 9, NECA (an A2b agonist) is 50% protective in ischemia alone. In conditions where ischemia and reperfusion are involved, the combination therapy containing A2b agonist and monoclonal antibody ⁽⁷¹⁻¹¹⁰⁾ would be very useful.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method of treating an ischemic reperfusion injury of a subject comprising:
administering to the subject a therapeutically effective amount of at least one of an adenosine receptor agonist or a protein kinase C activator and a therapeutically effective amount of a complement inhibitor.

2. The method of claim 1, the therapeutically effective amount of the adenosine receptor agonist or the protein kinase C activator being an amount effective to mitigate ischemic damage of the reperfused tissue.

3. The method of claim 2, the therapeutically effective amount of complement inhibitor being an amount effective to inhibit complement activation.

4. The method of claim 1, the adenosine receptor agonist comprising an A2b adenosine receptor agonist.

5. The method of claim 1, the complement inhibitor comprising a complement inhibiting antibody or fragment thereof.

6. The method of claim 5, the antibody comprising at least one of an anti-C3 antibody, anti-C3b antibody, anti-Ba antibody, anti-Bb antibody, anti-P antibody, anti-D antibody, anti-C5 antibody, anti-C5a antibody, anti-C6 antibody, anti-C7 antibody, anti-C8 antibody, and anti-C9 antibody.

7-9. (canceled)

10. The method of claim 5, the antibody reducing the level of properdin in the blood.

11. The method of claim 10, the reduced levels of properdin in blood decreasing levels of C3a, C5a, Bb, C5b-9 as a result of decreased alternative complement pathway activation during extracorporeal circulation.

12. (canceled)

13. A method of treating an ischemic reperfusion injury of a subject comprising:
administering to the subject a therapeutically effective amount of a A2b adenosine receptor agonist and a therapeutically effective amount of a complement inhibitor.

14. The method of claim 13, the therapeutically effective amount of the A2b adenosine receptor agonist being an amount effective to mitigate ischemic damage of the reperfused tissue.

15. The method of claim 13, the therapeutically effective amount of complement inhibitor being an amount effective to inhibit complement activation.

16. The method of claim 13, the complement inhibitor comprising a complement inhibiting antibody or fragment thereof.

17. The method of claim 16, the antibody comprising at least one of an anti-C3 antibody, anti-C3b antibody, anti-Ba antibody, anti-Bb antibody, anti-P antibody, anti-D antibody, anti-C5 antibody, anti-C5a antibody, anti-C6 antibody, anti-C7 antibody, anti-C8 antibody, and anti-C9 antibody.

18-20. (canceled)

21. The method of claim 16, the antibody reducing the level of properdin in the blood.

22. The method of claim 16, the reduced levels of properdin in blood decreasing levels of C3a, C5a, Bb, C5b-9 as a result of decreased alternative complement pathway activation during extracorporeal circulation.

23. A method of treating an ischemic reperfusion injury of a subject comprising:
administering to the subject a therapeutically effective amount of a A2b adenosine receptor agonist and a therapeutically effective amount of an anti-complement antibody.

24. The method of claim 23, the therapeutically effective amount of the A2b adenosine receptor agonist being an amount effective to mitigate ischemic damage of the reperfused tissue.

25. The method of claim 24, the antibody comprising at least one of an anti-C3 antibody, anti-C3b antibody, anti-Ba antibody, anti-Bb antibody, anti-P antibody, anti-D antibody, anti-C5 antibody, anti-C5a antibody, anti-C6 antibody, anti-C7 antibody, anti-C8 antibody, and anti-C9 antibody.

26-28. (canceled)

29. The method of claim 23, the antibody reducing the level of properdin in the blood.

30. (canceled)
